(19) United States
(12) Patent Application Publication
(10) Pub. No.: US 2008/0248590 A1
(43) Pub. Date: Oct. 9, 2008

(54) Device For Carrying Out A Biological Assay

(75) Inventors: Anja Gulliksen, Oslo (NO); Lars Anders Solli, Oslo (NO); Frank Karlsen, Klokkarstua (NO)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Norchip AS, Klokkarstua (NO)

(21) Appl. No.: 11/791,401

(22) PCT Filed: Nov. 25, 2005
(86) PCT No.: PCT/GB05/04524

§ 371 Date: Nov. 16, 2007

(30) Foreign Application Priority Data
Nov. 26, 2004 (GB) 0426082.4

(51) Int. Cl. G01N 33/543; B01J 19/00; B29C 65/00; C12M 1/00

(52) U.S. Cl. 436/518; 422/68.1; 435/287.2; 156/60

(57) ABSTRACT

An integrated lab-on-a-chip device for carrying out an assay to detect the presence of a biological molecule in a fluid sample, the device comprising: (a) an inlet for a fluid sample; (b) one or more reaction sites each in fluid communication with the inlet; (c) one or more reagent reservoir systems each containing reagents required for an assay to detect a biological molecule, the reagents being arranged sequentially in each reservoir system in the order in which they are required for the assay and separated from one another by a fluid.

### FIELD OF THE INVENTION

The invention is concerned with specific detection of biological molecules and, in particular, a lab-on-a-chip device for carrying out an assay to detect the presence of a biological molecule in a sample.

### BACKGROUND TO THE INVENTION

There is considerable interest in the development of simplified assay systems for detection of biological molecules which allow an unskilled user to perform complex assay procedures without undue error. Moreover, there is a great deal of interest in the development of contained assay systems which require minimal handling of liquid reagents and which can be automated to allow the assay procedure to be performed with minimal intervention from the user, and preferably also miniaturized to provide a convenient system for point-of-care testing. This is particularly relevant in the healthcare field, especially diagnostics, where there is an increasing need for biological assay systems which can be efficiently and safely operated within the doctor's surgery, the clinic, the veterinary surgery or even in the patient's home or in the field.

Microfabricated “lab-on-a-chip” devices are an attractive option for carrying out contained biological reactions requiring minimal reagent handling by the user and also permit the use of small sample volumes, a significant advantage for biological reactions which require expensive reagents.

One such device, for carrying out a polymerase chain reaction (PCR) followed by a detection step is disclosed in U.S. Pat. No. 5,674,742. Lamb wave pumps are used to transport DNA primers, polymerase reagents and nucleotide reagents from three separate storage chambers into a single reaction chamber as and when required to carry out a PCR process, with the temperature of the reaction site being cycled as required.

Another microfabricated device, for carrying out a chemical reaction step followed by an electrophoresis separation step, is disclosed in Analytical Chemistry 1994, 66, 4127-4132. Etched structures in a silicon substrate covered by a glass plate provide a reaction chamber and connections to buffer, analyte, reagent and analyte-waste reservoirs, as well as an electrophoresis column connected to a waste reservoir.

There is also considerable interest in development of medium-to-high throughput assay systems which allow detection of multiple target molecules in parallel. In the field of nucleic acid detection there has been much progress in the development of microarray-based systems for parallel processing and detection of multiple nucleic acid targets. There is much interest in applying microarray techniques, and the associated advantages of processing multiple reactions in parallel, to the detection of multiple non-nucleic acid target molecules, for example polypeptides.

Many biological assays are based on specific binding interactions between biological molecules and ultimately result in the generation of a signal or label which can be directly or indirectly detected, with the amount of signal or label detected being directly related to the amount, of the biological molecule present in the sample under test. A characteristic of such assays is that they require multiple, sequential reagent addition steps, possibly with intermediate washing steps. A difficulty faced in transferring such assays to a contained assay system, such as a lab-on-a-chip device, is in providing means for achieving this sequential addition of reagents in a pre-determined order.

The applicant's published International application WO 02/046464 describes a ligand detection assay based on real-time amplification of a nucleic acid marker. This method combines the specificity of binding assays, particularly immunoassays, with the sensitivity of nucleic acid amplification and can be used for real-time quantitative measurement. This assay, referred to by the inventors as immuno-real time amplification or “IMRAMP”, is particularly useful for detection of ligands which are present in very low amounts in complex test samples. Therefore, it is a desirable goal to be able to perform the IMRAMP assay in a contained device, and preferably a microfabricated device.

The present inventors have now developed a lab-on-a-chip device for use in carrying out biological assays. The device may be pre-loaded with all the reagents required for the assay in such a manner that they can be added sequentially in the order required for optimal performance of the biological assay. Furthermore, in particular embodiments the device may be adapted for detection of multiple different target molecules in a single sample in parallel. The device is suitable for, although not limited to, carrying out the IMRAMP assay technique described in WO 02/046464.

### SUMMARY OF THE INVENTION

Therefore, in a first aspect the present invention provides an integrated lab-on-a-chip device for carrying out an assay to detect the presence of a biological molecule in a fluid sample, the device comprising:

(b) one or more reaction sites each in fluid communication with the inlet;
(c) one or more reagent reservoir systems each containing reagents required for an assay to detect a biological molecule, the reagents being arranged sequentially in each reservoir system in the order in which they are required for the assay and separated from one another by a fluid;

wherein each of the one or more reaction sites is in fluid communication with a separate reagent reservoir system, whereby the reagents in each reagent reservoir system can be sequentially introduced into the reaction site in fluid communication therewith when the device is in use.

The device can be used on millilitre sample volumes for routine diagnostics. The device relies on certain reagents being pre-loaded, as discussed further below.

At least some of the components of the device are preferably microfabricated. Preferably, the reaction site(s) and the reagent reservoir system are microfabricated and integrated, meaning that they are formed on a common substrate.

The device will typically further comprise (d) means for sequentially introducing the reagents in each reagent reservoir system into the reaction site in fluid communication therewith. This means may be integrated with the other components of the device or the means may be separate or external, in which case the device may be supplied as a component of an apparatus comprising the device and separate means for sequentially introducing the reagents in each reagent reservoir system into the reaction site in fluid communication therewith. In either embodiment the means preferably comprises a pump or micropump. Alternatively, the means may be air pressure, preferably produced by a syringe or a variable volume chamber. The variable volume chamber may typically comprise a flexible membrane overlying a hollow recess in the underlying device.

As an alternative to the use of pumps or air pressure controlled movement of liquid reagents through the reagent reservoir system may be achieved by electrowetting actuation. Electrowetting actuation is a type of microfluidic manipulation which uses electrical fields to directly manipulate discrete droplets of fluid. In order to facilitate electrowetting actuation the device may include control electrodes to control the movement of liquid droplets and electrode connections to an external electrical power supply.

The device will typically further comprise (e) a waste unit or chamber in fluid communication with each of the reaction sites. Preferably the waste unit or chamber is microfabricated and preferably integrated with the other components of the device.

The device comprises one or more reaction sites and one or more reagent reservoir systems. Each one of the reaction sites is in fluid communication with one of the reagent reservoir systems. The reaction sites are all in fluid communication with a common inlet for input of a liquid sample, but are preferably not in fluid communication with other reaction sites by any means other than via fluid connections to the sample inlet. Should it be necessary, fluid flow from the inlet to each of the reaction sites may be controlled by the optional inclusion of valves. Depending on the design of the assay, and the construction of the device such control valves may not be necessary of it is possible to achieve controlled transport of the sample to the reaction samples without the use of valves.

The reagent reservoir systems are preferably separate, meaning that they are not in direct fluid communication with each other. This provides an advantage in parallel detection of, multiple different target molecules, since it minimises the possibility of cross-contamination between the detection assays carried out at each reaction site. Should it be necessary, fluid flow from a reagent reservoir to the reaction site in fluid communication therewith may be controlled by the optional inclusion of a valve.

The device may include at least 10, at least 20, at least 200, at least 2000 or even at least 20 000 reaction sites, each being in fluid communication with a separate reagent reservoir system.

Each reagent reservoir system is pre-loaded with reagents required for carrying out a detection assay in the associated reaction site. It is an important feature of the device of the invention that two or more or all of the reagents required to perform the assay are loaded into a common reagent reservoir. In devices with multiple reagent reservoirs and reaction sites the assay taking place in or at each of the individual reaction sites need not be the same, and preferably at least two different assays for different target molecules will be carried out in a single device. Typically, the device will be assembled such that multiple different target molecules can be detected, with the assays for the different molecules taking place in different reaction sites. Other reaction sites may be set up to perform positive or negative control assays.

The reagents are pre-loaded into each reagent reservoir in the order of addition to the reaction site, the order being pre-determined by the nature of the assay. The reagent to be added to the reaction site first in the assay is located most proximal to the reaction site.

The individual reagents in the reagent reservoir system are separated from each other by fluid gaps. The fluid may be any inert fluid, meaning any fluid which does not react with the reagents which it separates. The fluid prevents the reagents from mixing during storage, ensuring that the reagents are kept separate, and may also prevent the reagents from mixing during operation of the device, unless it is desired for two or more reagents to be mixed prior to addition to a reaction site. The most preferred fluids are inert gases, with air being particularly preferred.

If two liquid reagents are to be mixed prior to addition to the reaction site then the device may include a mixing unit to improve mixing of the liquid reagents. The mixing unit may be integrated with, or form part of the reagent reservoir system.

When the device includes multiple reagent reservoir systems each in fluid connection with an associated reaction site then it may include one single means for simultaneously sequentially introducing the reagents in each of reagent reservoir system into the associated reaction site, e.g. one single pump or syringe controlling fluid flow in all the reagent reservoir systems. Alternatively, each reagent reservoir may have associated therewith a separate means for sequentially adding the reagents, or the device may include more than one means, each associated with a subset or group of the reagent reservoirs. Where the means for sequentially introducing reagents is electrowetting then any convenient arrangement of electrodes can be used to control fluid in the reagent reservoirs separately or in any combination.

The reagent reservoirs may have any suitable shape and configuration but will typically be in the form of channels or conduits. The channels or conduits may be curved, arcuate or convoluted, and will typically be substantially sinuate, although substantially linear channels or conduits may also be used. Curved, arcuate, convoluted or sinuate reservoirs are advantageous in that they permit longer reservoirs containing multiple different reagents to be fitted into the device.

Each reagent reservoir contains at least two, and preferably at least three different liquid reagents each separated by a fluid, and may contain as many different liquid reagents as are required to carry out a complete biological assay. The term “reagents” as used herein includes but is not limited to enzymes, reaction buffers, enzyme substrates or cofactors, receptors having specific binding activity etc, also encompasses buffers used to perform intermediate washing steps between additions of components of the assay system. The inclusion of such washing steps will be familiar to those skilled in the art of assays for biological molecules. The precise chemical nature or composition of the reagents loaded into the device is generally not material to the invention.

The device may still further be supplied with one or more pre-loaded reagents in solid form. Suitable solid forms include, for example, freeze dried or lyophilised reagents. Certain assay reagents, typically enzymes, may be more stable if they are stored in freeze dried form and reconstituted immediately prior to use. One or more reagents in solid form may be incorporated into the reagent reservoir system. In one embodiment solid reagents may be stored in the main reagent reservoir, or in storage chambers in fluid communication with the main reagent reservoir, enabling a suitable fluid, such as a reaction buffer or water, to be added to reconstitute the solid reagent, and the resulting reconstituted reagent to be added in correct sequence with further liquid reagents stored in the main reagent reservoir.

The reaction sites included in the device may be of any suitable shape or form. Typically the reaction sites will be in the form of chambers or channels or parts thereof in fluid communication with the reagent reservoir-systems. Optionally valves may be provided between the reaction sites and the reagent reservoir systems and these may operate to control flow of sample into the reaction site from the inlet.

A reaction site and an associated reagent reservoir system may be integrated in a single common channel. In this arrangement the reaction site may be identified as the site within the channel where the assay for detection of the target molecule takes place. In embodiments wherein capture receptors are fixed, retained or immobilised at the reaction site then the reaction site will be defined/identified by the presence of the capture receptor.

Depending on the nature of the assay carried out in the device it may be advantageous or necessary to maintain a particular temperature above ambient temperature in a component of the device, or to vary the temperature of a particular component of the device during performance of the assay. The device may therefore further include heating means for supplying heat to and controlling the temperature in a component of the device, for example the reaction sites, mixing units, areas of the reagent reservoir system, etc. The heating means may be integrated with the other components of the device. Suitable heating means include, for example, Peltier elements.

The device of the invention may be adapted to carry out essentially any assay for detection of a biological molecule wherein it is required or desired to add multiple different reagents sequentially in a pre-determined order and/or to keep the reagents separate during storage and/or operation of the device.

By “biological molecule” is meant any molecular component of a biological system, or any molecule having an effect on a biological system, which it is desired to detect. This includes, inter alia, polypeptides, proteins, amino acids, sugars, complex carbohydrates, nucleic acids, nucleotides, multi-subunit proteins, aggregates or complexes of biological molecules, hormones, and also other small synthetic or naturally occurring molecules which affect biological systems, e.g. drugs, pro-drugs, environmental contaminants etc.

The device is particularly useful for carrying out biological binding assays. The features of biological binding assays are generally well known to those skilled in the art biological assay design, including diagnostics. In particular, the device may be adapted to perform all forms of sandwich binding assays, for example sandwich immunoassays analogous to ELISA assays.

A sandwich ELISA generally requires two receptors (e.g. antibodies) that are directed against a particular target molecule to be detected (e.g. an antigen). One receptor is immobilised onto a solid support such as a planar surface, a bead or the wells of a microtiter plate. Test samples suspected of containing the molecule to be detected are then added and incubated for sufficient time to allow the target molecule to bind to the receptors immobilised on the solid surface. After washing to remove unbound reagents a second receptor is added to the wells. This second receptor binds to the immobilized target molecule completing the sandwich. The second receptor is then detected by a suitable means in order to provide an indication of the amount of molecule present in the sample. Various different methods providing different assay read-outs can be used to detect the second receptor. Typical read-outs may involve, for example, detection of fluorescent labels, enzymatic reactions generating a colorimetic signal, electrochemical (e.g. amperometric) detection, amplification of a nucleic acid label and detection of the amplification products by real-time PCR or IMRAMP.

In other types of ELISA the test sample may be added directly to the solid support and incubated to allow test molecules present in the sample to become bound to the solid support. This type of assay is similar to the sandwich assay but does not require the first antibody, instead the test molecule is coated directly onto the solid support.

When such ELISA-type binding assays are performed in the device of the invention the reaction sites of the device may provide the solid support on which the assay takes place.

Typical binding assays require a “capture” receptor capable of specifically binding to the molecule to be detected. The capture receptor may be essentially any type of specific binding agent capable of specifically binding to the target molecule of interest. Suitable receptors include, but are not limited to, naturally occurring or recombinant biological binding agents such as, for example, antibodies or fragments thereof such as F(ab′)₂ fragments, scAbs, Fv and scFv fragments etc., nucleic acids, lectins, all types of ligand-binding receptors, such as hormone receptors, cytokine receptors etc., nucleic acid binding proteins and aptamers.

Devices according to the invention adapted to perform binding assays may be pre-loaded with capture receptors specific for the target molecule(s) to be detected. The capture receptors will be located in the reaction site when the device is in use and will preferably be pre-loaded into the reaction site. However, it is not excluded that the capture receptors may be located in another part of the device, for example the reagent reservoir or a separate storage chamber, during manufacture and/or storage of the device and then moved into the reaction site immediately prior to use, i.e. prior to addition of the sample to be tested.

The capture receptors are preferably fixed or retained in the reaction site throughout the duration of the assay. The capture receptors may be immobilised in the reaction site, for example by covalent linkage or non-specific adsorption to an interior surface of the reaction site. This ensures that the capture receptors remain located within the reaction site during all sample and reagent addition steps, effectively confining the assay to the reaction site. This arrangement opens up the possibility of using assay formats wherein the signal providing the final read-out of the assay is generated in free solution (e.g. the IMRAMP assay, real-time immuno-PCR etc.), and more particularly allows multiple assays based on such read-outs to be carried out in parallel within the device. Such assays can be carried out in parallel if the signal—generating steps of each individual assay, and preferably all steps of the assay, are contained and kept separate within or at separate reaction sites not in direct fluid communication with each other, such as is the case using the device of the invention.

Capture receptors may be introduced into the reaction sites during or after assembly of the device. Techniques which may be used to locate the capture receptors at the reaction sites include, but are not limited to, electrowetting, biocompatible photolithography and laser ablation.

When the device of the invention is in use, liquid sample to be tested for the presence of the target molecule is introduced into the device at the inlet and communicated to the reaction sites. Fluid communication between the inlet hole and the reaction sites may be achieved via a common supply channel with branches to each of the reaction sites. The supply channel may also be in fluid communication with a waste unit or chamber, allowing excess sample to be communicated to waste and contained within the device.

The test sample to be tested for the presence of a particular biological molecule may be essentially any fluid sample it is desired to test for the presence of a ligand. It may be, for example, a clinical sample, an environmental fluid etc. For example, in the diagnostics field the test sample may comprise body fluids such as whole blood, serum, plasma, lymph, tears, urine, ascites, pleural effusion, etc. The sample may be diluted or concentrated prior to application to the device or it may be subject to pre-treatment steps to alter the composition, form or some other property of the sample. Pre-treatment steps may include, for example, cell lysis.

The read-out of the assay carried out in the device of the invention may be detected or measured using any suitable detection or measuring means. The detection means will vary depending on the nature of the read-out of the assay. For assays providing a fluorescent read-out the detection means may include a source of fluorescent light at an appropriate wavelength to excite the fluorophores in the reaction sites and means detect the emitted fluorescent light at the appropriate wavelength. The excitation light may be filtered using a bandwidth filter before the light is collimated through a lens. The same (e.g. Fresnel) lens may be used for focusing the illumination and collection of the fluorescence light. Another lens may be used to focus the fluorescent light onto the detector surface (e.g. a photomultiplier-tube). Fluorescent read-outs can also be detected using a standard fluorescent microscope fitted with a CCD camera and software. The invention also relates to an apparatus or system including a device according to the invention and a detection means as described herein.

The device of the invention is preferably microfabricated. Preferably the device is disposable. By the term “microfabricated device” as used herein is meant any device manufactured using processes that are typically, but not exclusively, used for batch production of semiconductor microelectronic devices, and in recent years, for the production of semiconductor micromechanical devices. Such microfabrication technologies include, for example, epitaxial growth (e.g. vapour phase, liquid phase, molecular beam, metal organic chemical vapour deposition), lithography (e.g. photo-, electron beam-, x-ray, ion beam-), etching (e.g. chemical, gas phase, plasma), electrodeposition, sputtering, diffusion doping and ion implantation. Although non-crystalline materials such as glass may be used, microfabricated devices are typically formed on crystalline semiconductor substrates such as silicon or gallium arsenide, with the advantage that electronic circuitry may be integrated into the device by the use of conventional integrated circuit fabrication techniques. Combinations of a microfabricated component with one or more other elements such as a glass plate or a complementary microfabricated element are frequently used and intended to fall within the scope of the term microfabricated used herein. Also intended to fall within the scope of the term microfabricated are polymeric replicas made from, for example, a crystalline semiconductor substrate. The terms microfabricated and microfabricated device as used herein are also intended to encompass nanofabricated devices.

Fluidics is the science of liquid flow in, for example, tubes. For microfabricated devices, flow of a fluid through the one or more sets of micro or nano sized reaction sites is typically achieved using a pump such as a syringe, rotary pump or precharged vacuum or pressure source external to the device. Alternatively, a micro pump or vacuum chamber, or lamb wave pumping elements may be provided as part of the device itself. Other combinations of flow control elements including pumps, valves and precharged vacuum and pressure chambers may be used to control the flow of fluids through the reaction sites. Other mechanisms for transporting fluids within the system include electro-osmotic flow and electrowetting.

The device or at least a master version thereof will typically be formed from or comprise a semiconductor material, although dielectric (e.g. glass, fused silica, quartz, polymeric materials and ceramic materials) and/or metallic materials may also be used. Examples of semiconductor materials include one or more of: Group IV elements (i.e. silicon and germanium); Group III-V compounds (e.g. gallium arsenide, gallium phosphide, gallium antimonide, indium phosphide, indium arsenide, aluminium arsenide and aluminium antimonide); Group II-VI compounds (e.g. cadmium sulphide, cadmium selenide, zinc sulphide, zinc-selenide); and Group IV-VI compounds (e.g. lead sulphide, lead selenide, lead telluride, tin telluride). Silicon and gallium arsenide are preferred semiconductor materials. The device may be fabricated using conventional processes associated traditionally with batch production of semiconductor microelectronic devices, and in recent years, the production of semiconductor micromechanical devices. Such microfabrication technologies include, for example, epitaxial growth (e.g. vapour phase, liquid phase, molecular beam, metal organic chemical vapour deposition), lithography (e.g. photo-, electron beam-, x-ray, ion beam-), etching (e.g. chemical, gas phase, plasma), electrodeposition, sputtering, diffusion doping, ion implantation and micromachining. Non-crystalline materials such as glass and polymeric materials may also be used.

Examples of polymeric materials include PMMA (Polymethyl methylacrylate), COC (Cyclo olefin copolymer), polyethylene, polypropylene, PL (Polylactide), PBT (Polybutylene terephthalate) and PSU (Polysulfone), including blends of two or more thereof. The preferred polymer is PDMS or COC.

The device will typically be integrally formed. The device may be microfabricated on a common substrate material, for example a semiconductor material as herein described, although a dielectric substrate material such as, for example, glass or a ceramic material could be used. The common substrate material is, however, preferably a plastic or polymeric material and suitable examples are given above. The device may preferably be formed by replication of, for example, a silicon master.

The advantages of using plastics instead of silicon-glass for miniaturized structures are many, at least for biological applications. One of the greatest benefits is the reduction in cost for mass production using methods like microinjection moulding, hot embossing and casting. A factor of a 100 or more is not unlikely for complex structures. The possibility to replicate structures for multilayered mould inserts gives a great flexibility of design freedom. Interconnection between the micro and macro world are in many cases easier because one has the option to combine standard parts normally used. Different approaches can be used for assembly techniques, like e.g. US-welding with support of microstructures, laser welding, gluing and lamination. Surface modification may also be included. For miniaturized structures addressed for biological analysis, it is important that the surface is biocompatible. By utilizing plasma treatment and plasma polymerization a flexibility and variation of assortment can be adapted into the coating. Chemical resistance against acids and bases are much better for plastics than for silicon substrates that are easily etched away. Most detection methods within the biotechnological field involve optical measurements. The transparency of plastic is therefore a major feature compared to silicon that is not transparent. Polymer microfluidic technology is now an established yet growing field within the Lab-on-a-chip market.

For a silicon or semiconductor master, it is possible to define by, for example, etching or micromachining, one or more of variable volume chambers, microfluidic channels, reaction sites and fluid interconnects in the silicon substrate with accurate microscale dimensions. A plastic replica may then be made of the silicon master. In this manner, a plastic substrate with an etched or machined microstructure may be bonded by any suitable means (for example using an adhesive or by heating) to a cover.

The optional valves used in the device may take any convenient form. For example, the valves may simply regulate flow along a conduit or channel connecting two units. A piston-like member may be provided which can be raised or lowered in a hole in a conduit or channel by the action of a pin device.

In a further aspect the invention provides a method for the manufacture of an integrated lab-on-a-chip device for carrying out an assay to detect the presence of a biological molecule as described herein, which method comprises:

A) providing a substrate having an inlet recess, one or more reaction site recesses and one or more reagent reservoir system recesses in a surface thereof;
B) providing a cover; and
C) bonding the cover to the substrate to create (a) the inlet, (b) one or more reaction sites and (c) one or more reagent reservoir systems, each being defined by the respective recess in said surface and the adjacent surface of the cover.

The term recess as used herein is also intended to cover a variety of features including, for example, grooves, slots, holes, trenches and channels, including portions thereof.

The method may further comprise the step of introducing reagents required for an assay to detect the presence of a biological molecule into at least one reagent reservoir system either before or after bonding the cover to the substrate. This further step may comprise introducing two or more liquid reagents into at least one reagent reservoir system either before or after bonding the cover to the substrate, wherein the liquid reagents are separated by a fluid.

The method may still further comprise the step of introducing capture binding receptors into at least one reaction site either before or after bonding the cover to the substrate.

Reagents may be pre-loaded into the device before or after bonding of the cover to the substrate. In one embodiment the cover may include holes or cavities overlying areas in the device into which it is required to pre-load reagents. After bonding of the cover to the substrate reagents may be introduced into chambers or channels defined by the substrate and the internal surface of the cover via these holes or cavities. This can be achieved, for example, with the use of a spotter to add controlled volumes of reagent. After addition of the reagents the holes or cavities may be sealed with an appropriate sealing material.

The substrate may be formed from silicon, for example, and the overlying cover from glass, for example. In this case, the glass cover is preferably anodically bonded to the silicon substrate, optionally through an intermediate silicon oxide layer formed on the surface of the substrate. The recesses in the silicon may be formed using reactive-ion etching. Other materials such as polymeric materials may also be used for the substrate and/or cover. Such materials may be fabricated using, for example, a silicon replica. Alternatively, the device may be fabricated by structuring of mould inserts by milling and electro-discharge machining (EDM), followed by injection moulding of the chip parts, followed by mechanical post-processing of the polymer parts, for example drilling, milling, debarring. This may subsequently be followed by insertion of the filter, solvent bonding, and mounting of fluidic connections.

Examples of polymeric materials include PMMA (Polymethyl methylacrylate), COC (Cyclo olefin copolymer) polyethylene, polypropylene, PL (Polylactide), PBT (Polybutylene terephthalate) and PSU (Polysulfone), including blends of two or more thereof. COC is preferred.

Preferably, and in particular if optical observations of the contents of the cell are required, the overlying cover is made of an optically transparent substance or material, such as glass, Pyrex or COC.

Combinations of a microfabricated component with one or more other elements such as a glass plate or a complementary microfabricated element are frequently used and intended to fall within the scope of the term microfabricated used herein.

Part or all of the substrate base may be provided with a coating of thickness typically up to 1 μm, preferably less than 0.5 μm. The coating is preferably formed from one or more of the group comprising polyethylene glycol (PEG), Bovine Serum Albumin (BSA), tweens and dextrans. Preferred dextrans are those having a molecular weight of 9,000 to 200,000, especially preferably having a molecular weight of 20,000 to 100,000, particularly 25,000 to 75.000. for example 35,000 to 65,000). Tweens (or polyoxyethylene sorbitans) may be any available from the Sigma Aldrich Company. PEGs are preferred as the coating means, either singly or in combination. By PEG is embraced pure polyethylene glycol, i.e. a formula HO—(CH₂CH₂O)ₙ—H wherein n is an integer whereby to afford a PEG having molecular weight of from typically 200-10,000, especially PEG 1,000 to 5,000; or chemically modified PEG wherein one or more ethylene glycol oligomers are connected by way of homobifunctional groups such as, for example, phosphate moieties or aromatic spacers. Particularly preferred are polyethylene glycols known as FK108 (a polyethylene glycol chain connected to another through a phosphate); and the PEG sold by the Sigma Aldrich Company as product P2263. The above coatings applied to the surfaces of the chamber, inlets, outlets, and/or channels can improve fluid flow through the device. In particular, it has been found that the sample is less likely to adhere or stick to such surfaces. PEG coatings are preferred.

For a silicon or semiconductor master, it is possible to define by, for example, etching or micromachining, one or more of variable volume chambers, microfluidic channels, reaction sites and fluid interconnects in the silicon substrate with accurate microscale dimensions (deep reactive-ion etching (DRIE) is a preferred technique). A plastic replica may then be made of the silicon master. In this manner, a plastic substrate with an etched or machined microstructure may be bonded by any suitable means (for example using an adhesive or by heating) to a cover thereby forming the enclosed chamber(s), inlet(s), outlet(s) and connecting channel(s).

The device comprises a substrate with the desired microstructure formed in its upper surface. The substrate may be silicon, for example, or a plastic substrate formed by replication of a silicon master. The substrate is bonded at its upper surface to a cover, thereby defining a series of units/chambers, inlets, outlets, and/or channels. The cover may be formed from plastic or glass, for example. The cover is preferably transparent and this allows observation of the fluid. In general, the device is preferably fabricated by deep reactive-ion etching (DRIE) of silicon for high aspect ratio constrictions, followed by anodic bonding of a glass cover. Alternatively, the device may be fabricated by structuring of mould inserts by milling and electro-discharge machining (EDM), followed by injection moulding of the chip parts, followed by mechanical post-processing of the polymer parts, for example drilling, milling, debarring. This may subsequently be followed by insertion of the filter, solvent bonding, and mounting of fluidic connections.

The invention also extends to a method of carrying out an assay for detection of a biological molecule using the device according to the invention as described herein, the method comprising loading a sample to be tested into the device via the inlet, communicating the sample to the one or more reaction sites and sequentially adding the reagents pre-loaded in at least one reagent reservoir system to the reaction site in fluid communication therewith in the order in which they are stored in the reagent reservoir system.

The method may include a further step of detecting the read-out of the assay carried out at one or more of the reaction sites.

### BRIEF DESCRIPTION OF THE DRAWINGS

FIG. 1 is a schematic illustration of a device according to the invention.

FIG. 2 is a schematic illustration of a further device according to the invention.

FIG. 3 is a schematic illustration of a reaction site and reagent reservoir system for use in a device according to the invention, illustrating the order of reagent pre-loading for an exemplary assay.

FIG. 4 is a schematic illustration of reagent pre-loading in a device according to the invention.

FIG. 5 is a schematic illustration of mechanisms for mixing of reagents in a device according to the invention.

### DESCRIPTION OF PREFERRED EMBODIMENTS

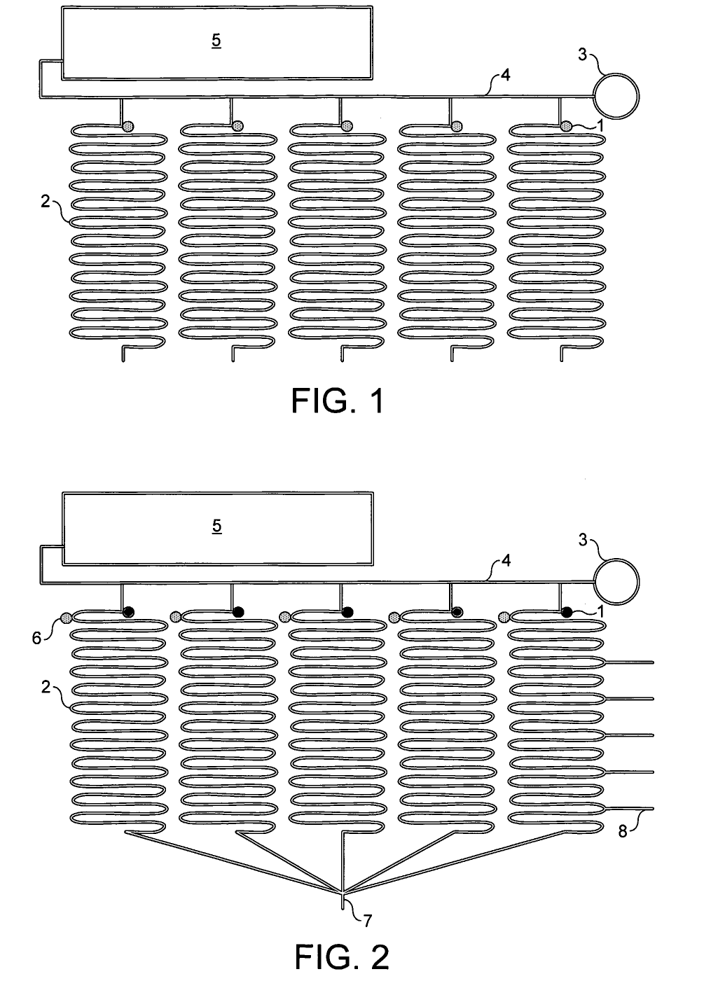

Referring to the drawings, FIG. 1 shows a device according to the invention comprising several reaction sites 1 each in fluid communication with a separate reagent reservoir system 2. The sample to be tested is applied at the inlet hole 3 and is communicated to each of the reaction sites via a supply channel 4. In this embodiment the reagent reservoir systems are formed of sinuate channels. The supply channel is in fluid communication with a waste chamber 5.

FIG. 2 shows an alternative device according to the invention. This embodiment is substantially similar to the device illustrated in FIG. 1 but further includes valves 6 located between each of the reaction sites and the reagent reservoir in fluid communication therewith. The valves will be opened for air when the sample is loaded. The sample will then fill the reaction sites until it reaches the valve. The valves are then closed. The rest of the sample will be drained into the waste unit or chamber 5. If required, such valves may be included in the embodiment shown in FIG. 1.

In the embodiment shown in FIG. 2, each of the reagent reservoirs is in fluid communication, at the end distal from the reaction site, with a common means for supplying air pressure 7. This means may be, for example, a syringe, pump or micropump or a variable volume chamber. The means may be integrated into the device or may be a separate component. In the embodiment shown in FIG. 2 air pressure is supplied from the back of the chip. When the device is in use air pressure is applied to move reagents through the reagent reservoir system such that they are sequentially brought into contact with the reaction site. Liquid reagents flushed out of the reaction site are collected in the waste unit it chamber, which is integrated with the other components of the device. This common means for applying air pressure may also be included in the device illustrated in FIG. 1.

The device illustrated in FIG. 2 still further includes support channels 8 which may be used for pre-loading of reagents into the reagent reservoir system.

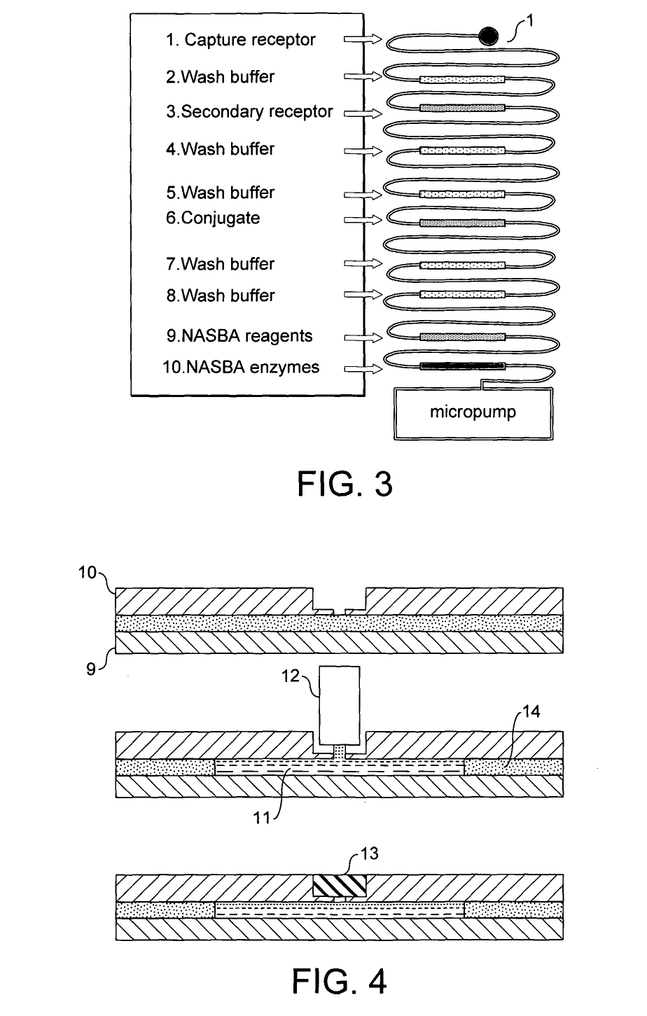

FIG. 3 illustrates a reagent reservoir system for use in the device according to the invention. This system may be used in conjunction with all embodiments of the device described herein. The embodiment illustrated is a reagent system pre-loaded with reagents for carrying out an immuno real-time NASBA amplification reaction, as described in WO 02/046464. However, it will be understood that this is by way of example only and the invention is not limited to devices pre-loaded with reagents for this specific assay method. Referring to FIG. 3, a capture receptor is fixed, retained or immobilised at the reaction site 1. Reagents are pre-loaded into the reagent reservoir in the order they are required for the assay taking place at the reaction site. In this particular example the reagents are, in order, a wash buffer, secondary receptors, wash buffer, was buffer, IMRAMP conjugate comprising a further receptor linked to a nucleic acid label, wash buffer, wash buffer, NASBA reagents and NASBA enzymes. Each of the discrete reagents is separated by a gap or plug of fluid, for example air. The distal end of the reagent reservoir system is connected to a means for sequentially introducing the discrete reagents into the reaction site. In this embodiment the means is shown as a micropump. However, any of the means described herein can be used in conjunction with the same reagent reservoir system.

When a device incorporating this reagent reservoir system is in use, any target molecules present in the sample under test will attach to the capture receptors at the reaction site 1. After the target molecules are bound to the capture receptors, it is possible to perform an assay to detect the amount of target molecules present using the reagents stored in the reservoir system. In the case of an IMRAMP assay (method described generally in WO 02/046464, the contents of which are incorporated herein in their entirety by reference) after binding of the target molecule to the capture receptor, a washing buffer (number 2) flushes over the capture receptor and target complexes at the reaction site (number 1). This may be done by pushing the wash buffer through the reaction site using suitable means, such as air pressure from the back of the device (in devices such as those shown in FIGS. 1 and 2 all the reagents flushed out of the reaction site will end up in the waste unit or chamber). By doing this all the preloaded reagents in the device are moving against the capture receptors at number 1 in sequence. A secondary receptor (number 3) can be applied in the same manner as for the wash buffer, being preloaded in the device. After binding of the secondary receptor, the complexes are washed again with wash buffer (number 4 and 5) to get rid of excess unbound reagents. An IMRAMP conjugate (number 6) will then be added to the complexes at the reaction site (number 1). The reaction site is then washed (number 7 and 8) again to get rid of excess reagents before the NASBA reagents (number 9) and enzymes (number 10) are introduced into the reaction site. The NASBA reaction mixture (both reagents and enzymes) is incubated over the activated target-receptor complexes now present at the reaction site in order to start the detection phase of the IMRAMP reaction. Most preferably detection is carried out by real-time NASBA using molecular beacons, as described in WO 02/046464. By using optimal detection systems and software, it is possible to measure fluorescence light from detection of NASBA amplification products generated at the reaction site. Details of the buffers, reagents, enzymes, probes, detection apparatus etc required for real-time NASBA are described in detail in WO 02/046464.

FIG. 4 illustrates a method for pre-loading of reagents into a device according to the invention. This method may be used in conjunction with all embodiments of the device described herein. A substrate 9 including recesses corresponding to the reaction sites and reagent reservoir systems to be included in the final device is provided. A cover 10 is then bonded to the substrate to create the reaction sites and reagent reservoirs, these being defined by the corresponding recesses in the substrate and the internal surface of the cover. The cover includes holes or cavities overlying areas where reagents are to be pre-loaded into the device. After bonding of the cover to the substrate reagents 11 may be loaded into the reagent reservoirs defined by the substrate and the cover by means of these holes or cavities. The appropriate volume of reagent may be dispensed through the hole or cavity with the use of a spotter 12. The device is then sealed by plugging the hole or cavity with a suitable sealing material which prevents leakage of the reagent 13. The areas of loaded reagents 11 are typically bounded by air 14.

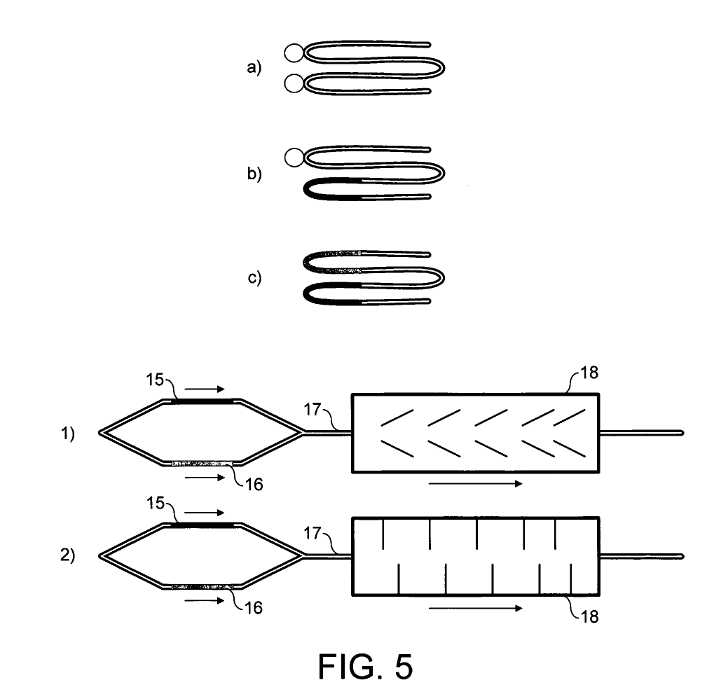

Several solutions are proposed for mixing of pre-loaded reagents within the device, as illustrated in FIG. 5. There are various technical reasons why it may be desirable to mix reagents in situ within the device. For example, if the assay requires the use of enzymes then the enzymes may have to be stored separately from other reaction components in order to maintain enzyme activity. This is the case with reagents for NASBA, which should be stored separately from the remaining reaction components required for the NASBA reaction in order to retain activity. The NASBA enzymes and remaining reaction components must, however, be mixed in order to allow the NASBA reaction to proceed. Mixing of reagents may take place either be performed where the reagents are loaded and stored or at the reaction site. Two agents requiring mixing may be stored on chip in the following conditions:
(a) Both reagents (e.g. NASBA reagents and enzymes) are dried on chip
(b) One of the reagents is dried on chip and one liquid solution
(c) Both reagents are liquids

The reagents in the dry state may have the best stability and may therefore be stored for a longer period.

For alternative 1, an additional liquid may to be loaded in the device for dissolving the dried reagents before they are moved to the reaction site. This additional liquid may be pre-loaded into the reagent reservoir system or may be stored in a separate buffer storage chamber in liquid communication with the reagent reservoir system. Mixing may thus occur by diffusion. The buffer storage chamber may be common to all the reagent reservoir systems in the device, i.e. a single storage chamber in liquid communication with each of the reagent reservoir systems.

For alternative 2, the liquid reagent may be used as a solvent for the dried reagent. Mixing may again occur by diffusion.

For alternative 3, the two liquids reagents have to be mixed together. FIG. 5 illustrates two mixing unit arrangements which may be used to mix two liquid reagents. In the first arrangement the two liquid reagents are stored in separate channels 15,16 which are in fluid communication at one end with a common supply channel 17, through which the reagents are introduced into a mixing unit 18. In one embodiment the mixing unit 18 may include a series of pairs of divergent baffles positioned against the flow of liquid through the mixing unit when in use. In a second embodiment the mixing unit 18 may incorporate a twisted channel. In further embodiments reagents may be mixed by electrowetting actuation.

In a particular preferred, but non-limiting, embodiment the invention provides a device as described herein which is pre-loaded with all of the reagents necessary to carry out detection of a target molecule by immuno real-time amplification and detection using antibodies as the capture and secondary receptors and using real-time NASBA based on use of molecule beacons probes to detect a nucleic acid label attached to the secondary antibody via formation of a molecular conjugate.

1. An integrated lab-on-a-chip device for carrying out an assay to detect the presence of a biological molecule in a fluid sample, the device comprising:
(a) an inlet for a fluid sample;
(b) one or more reaction sites each in fluid communication with the inlet;
(c) one or more reagent reservoir systems each containing reagents required for an assay to detect a biological molecule, the reagents being arranged sequentially in each reservoir system in the order in which they are required for the assay and separated from one another by a fluid;
wherein each of the one or more reaction sites is in fluid communication with a separate reagent reservoir system, whereby the reagents in each reagent reservoir system can be sequentially introduced into the reaction site in fluid communication therewith when the device is in use.

2. A device according to claim 1 which further includes (d) means for sequentially introducing the reagents in each reagent reservoir into the reaction site in fluid communication therewith.

3. A device according to claim 2 wherein the means for sequentially introducing the reagents in each reagent reservoir into the reaction site in fluid communication therewith is a micropump.

4. A device according to claim 2 wherein the means for sequentially introducing the reagents in each reagent reservoir into the reaction site in fluid communication therewith is a variable volume chamber.

5. A device according to claim 2 wherein the means for sequentially introducing the reagents in each reagent reservoir into the reaction site in fluid communication therewith is electrowetting.

6. A device according to claim 1, further comprising (e) a waste unit in fluid communication with each of the reaction sites.

7. A device according to claim 1, wherein at least one reagent reservoir system is pre-loaded with two or more different liquid reagents, each reagent being separated by a fluid.

8. A device according to claim 7 which further comprises (f) a mixing unit in fluid communication with at least one reagent reservoir system pre-loaded with two or more liquid reagents.

9. A device according to claim 1 wherein the fluid separating the reagents in at least one reservoir system is air.

10. A device according to claim 1 wherein each reagent reservoir system comprises a channel or conduit in fluid communication with a reaction site.

11. A device according to claim 10 wherein the channel or conduit is curved, arcuate, convoluted or sinuate.

12. A device according to claim 1 wherein at least one reagent reservoir system is pre-loaded with one or more reagents in solid form.

13. A device according to claim 1, wherein liquid sample introduced at the inlet is communicated to the reaction site(s) by electrowetting when the device is in use.

14. A device according to claim 1 wherein the assay is a biological binding assay.

15. A device according to claim 14, wherein at least one reaction site contains a capture binding receptor capable of specifically binding to a biological molecule to be detected.

16. A device according to claim 15, wherein the capture binding receptor is immobilised in the reaction site.

17. A device according to claim 15 wherein the capture binding receptor comprises an antibody, an F(ab′)₂ fragment, a single chain antibody fragment, an Fv fragment, a single chain Fv fragment, a nucleic acid, a lectin, a ligand-binding receptor, a hormone receptor, a cytokine receptor, a nucleic acid binding protein or an aptamer.

18. A device according to claim 15 wherein the reagent reservoir is pre-loaded with, in order of proximity to the reaction site, a reagent comprising secondary receptors capable of binding to the molecule to be detected, optionally a wash buffer, and one or more reagents which permit detection of the secondary receptors when bound to the molecule to be detected.

19. A device according to claim 18 wherein the reagents which permit detection of the secondary receptors comprise, in order of proximity to the reaction site, reagents capable of forming a reagent complex when added to the reaction site, which reagent complex comprises the secondary receptor linked to a nucleic acid label molecule, and reagents which permit detection of the nucleic acid label by amplifying a region of the nucleic acid and simultaneously detecting products of the amplification reaction in real-time.

20. A device according to claim 19, wherein the reagent reservoir is pre-loaded with, in order of proximity to the reaction site, a wash buffer, secondary receptors, a wash buffer, a conjugate comprising a nucleic acid label linked to a component capable of specifically binding to the secondary receptors, a wash buffer, reaction buffer for real-time NASBA detection of the nucleic acid label and enzymes for real-time NASBA detection of the nucleic acid label.

21. An apparatus for use in carrying out an assay to detect the presence of a biological molecule, the apparatus comprising a device according to claim 1.

22. An apparatus according to claim 21 which includes separate means for sequentially introducing the reagents in each reagent reservoir into the reaction site in fluid communication therewith.

23. A method for the manufacture of an integrated lab-on-a-chip device for carrying out an assay to detect the presence of a biological molecule as described in claim 1, which method comprises:
A) providing a substrate having an inlet recess, one or more reaction site recesses and one or more reagent reservoir system recesses in a surface thereof;
B) providing a cover; and
C) bonding the cover to the substrate to create (a) the inlet, (b) one or more reaction sites and (c) one or more reagent reservoir systems, each being defined by the respective recess in said surface and the adjacent surface of the cover.

24. A method according to claim 23, further comprising the step of introducing reagents required for an assay to detect the presence of a biological molecule into at least one reagent reservoir system either before or after bonding the cover to the substrate.

25. A method according to claim 24 which comprises introducing two or more different liquid reagents into at least one reagent reservoir system either before or after bonding the cover to the substrate, wherein the liquid reagents are separated by a fluid.

26. A method according to claim 23, further comprising the step of introducing capture binding receptors into at least one reaction site either before or after bonding the cover to the substrate.

27. A method of carrying out an assay for detection of a biological molecule using a device according to claim 1, the method comprising loading a fluid sample to be tested into the device via the inlet, communicating the sample to the one or more reaction sites and sequentially adding the reagents pre-loaded in at least one reagent reservoir system to the reaction site in fluid communication therewith in the order in which they are stored in the reagent reservoir system.
